(19) United States
(12) Patent Application Publication
(10) Pub. No.: US 2007/0298059 A1
(43) Pub. Date: Dec. 27, 2007

(54) Method for Identification and Verification of Products Containing Titanium Dioxide Pigment Particles

(76) Inventors: Peter Tiedemann, Leichlingen (DE); Siegfried Blumel, Ratingen-Eggerscheid (DE); Roland Herzog, Monheim (DE); Volker Schmitt, Leichlingen (DE)

Correspondence Address: LOCKE LORD BISSELL & LIDDELL LLP, 2200 ROSS AVENUE, SUITE 2200, DALLAS, TX, 75201-6776, US

(21) Appl. No.: 11/757,616

(22) Filed: Jun. 4, 2007

Related U.S. Application Data
(60) Provisional application No. 60/807,293, filed on Jul. 13, 2006.

(30) Foreign Application Priority Data
Jun. 23, 2006 (DE) 10 2006 029 284

(51) Int. Cl. A61K 8/29; B32B 27/04; C09C 1/36

(52) U.S. Cl. 424/401; 442/123; 106/437

(57) ABSTRACT

A method for marking titanium dioxide pigment particles, as well as a method for identifying and verifying products via the marked titanium dioxide pigment particles contained in these products. The marker substance(s) is/are added during titanium dioxide production, preferably during surface treatment, drying or steam-milling. Particularly suitable as marker substances are the lanthanides, yttrium, zinc, molybdenum, tungsten, germanium, tin and scandium. The method is used to verify authentic product, to identify product forgeries (product piracy), to track distribution channels, or to identify the pigment used in order to counter unjustified complaints. It can be used in the product segments: fibres for textiles, papers for documents, pharmaceuticals, and also for plastics and coatings.

### RELATED APPLICATION

This application claims the benefit of U.S. Provisional patent application Ser. No. 60/807,293 filed Jul. 13, 2006 and the benefit of DE 10 2006 029 284 filed Jun. 23, 2006.

### FIELD OF THE INVENTION

The invention relates to a method for identifying and verifying products containing titanium dioxide pigment particles by using marked titanium dioxide pigment particles. The invention further relates to a method for producing marked titanium dioxide pigment particles, as well as to the titanium dioxide pigment particles marked in this way.

### BACKGROUND OF THE INVENTION

So-called “product piracy” and fake products are widespread on the market and mean major financial losses for the manufacturers of the original products. There is consequently great interest in methods that can be used for unequivocally and reliably distinguishing original products from fake products. There are several approaches in this context.

One possibility consists in “marking” the original products. In this case, certain unequivocally detectable materials are added to the products, such as code-bearing microparticles or fluorescent, phosphorescent or radioactive substances. These substances can either be subsequently applied to the products externally, or added during production. For example, DE 100 83 295 T1 describes a method where a carpet product is provided with a marker substance by the marker substance being mixed into a customary protective fluid, and the protective fluid subsequently being applied to the carpet product as a protective layer. In another method, inks that are transparent in visible light are, for example, applied to the outside of documents, and their presence or absence is detected by means of ultraviolet or infrared fluorescence. In another application, a fluorescent dye is added to the plastic of Compact Discs for marking. Alternatively, the raw materials or semi-finished products used for product manufacture can also be marked. In the method according to U.S. Pat. No. 6,035,914, for example, fibres are mixed with a fluorescent dye in such a way that they display fluorescent, dichroic behaviour when irradiated with electromagnetic radiation of the appropriate wavelength. These fibres can be used in paper for producing bank notes, or laminated into plastic products, such as credit cards.

The markings on the products are usually not detectable without special analytical methods.

The methods for marking and subsequent identification must be optimised in relation to the respective application. They are often expensive and time-consuming to use. In some cases, the marker substance also presents a health hazard.

### SUMMARY OF THE INVENTION

The invention is a method for identification and verification of products via the titanium dioxide pigment particles contained in these products.

The method of the invention allows for marking a product containing titanium dioxide pigment particles, characterised in that the titanium dioxide pigment particles contain a quantity of at least one detectable marker substance.

The method of the invention allows for marking titanium dioxide pigment particles, characterised in that a quantity of at least one marker substance is used during production of the titanium dioxide particles, where the marker substance is detectable in a product containing the titanium dioxide particles.

The method of the invention is also a product containing titanium dioxide pigment particles, characterised in that the titanium dioxide pigment particles contain a quantity of at least one detectable marker substance.

### DESCRIPTION OF THE PREFERRED EMBODIMENT

The invention involves the addition of a marker substance, which can be used to identify the product, to the titanium dioxide pigment during the manufacturing of the product. The terms “product” and “end product” are used equivalently below. The invention encompasses the use of both one marker substance and several marker substances in pigment production.

As is generally known, titanium dioxide pigment is produced from iron-titanium ores, iron-titanium slags or corresponding heavy-mineral concentrates by the so-called sulphate process or the so-called chloride process. The pure titanium dioxide, also referred to as titanium dioxide base material, is conditioned with the help of certain additives in such a way that the pigmentary optical properties, e.g. brightness, tinting strength and tone, and also the photostability and the processing properties (flow properties, dispersion performance, etc.), are optimised for the respective application. Conditioning is performed by doping with suitable elements in the crystal lattice and/or by coating the surface with suitable inorganic and/or organic compounds.

A dense SiO2 coating, for example, leads to improved photostability of the TiO2 pigment. The pigment dispersibility in aqueous systems is, on the other hand, improved by final coating with a layer of hydrous aluminium oxide. The TiO2 pigment is surface-treated with silanes for easier incorporation in plastics. These methods are exhaustively described in the prior art (e.g. Ullmann's Encyclopedia of Industrial Chemistry, CD-ROM 2006, 7th Edition: “Inorganic Pigments, Chapter 2.1.3.4. Aftertreatment”) and well known to the person skilled in the art.

The impact of the additives on the physico-chemical behaviour, and thus the utility of the titanium dioxide pigment in its customary fields of application (paints and coatings, plastics, fibres, paper, etc.) is likewise known. It is therefore desirable that the marker substances used in the present invention do not alter the properties of the titanium dioxide pigment optimised for the intended field of application, but can nevertheless be detected in the end product with the help of suitable methods. Similarly, it is desirable that no negative effects on the properties of the end product occur, e.g. due to interactions with other raw material components.

It is desirable that the detection methods to be used for the marker substances yield unequivocal, reconstructable results. Examples of suitable methods include X-ray fluorescence analysis (XRF), atomic absorption spectroscopy (MS), atomic emission spectroscopy (flame AES, ICP-AES), neutron activation analysis (NM) and ICP mass spectrometry (ICP-MS).

The marker substance to be used according to the invention can be added both by doping in the crystal lattice and when coating the surface (hereafter: surface treatment). Marking during surface treatment is preferred for reasons of process engineering.

Different marker substances can be used in combination. Defined concentration ratios can be set in this context, such that the product is virtually identified by a marker substance code. Individual production batches can be distinguished from each other in this way. The combination of two or more marker substances leads to improved unequivocalness of identification and less risk of confusion of marked and unmarked products.

Suitable marker substances are, in principle, all elements that are not used anyway for the doping or surface treatment of the pigment, or are not present in the TiO2 as detectable contamination—originating from the ore or the slag, such as Cr, V or Nb—or do not occur as impurities or components in the end product. It is desirable that the marker substances not contain any colouring elements.

As the marker substances are at all events used in minimal concentrations, colouring elements can be used in cases where whiteness plays no decisive role in the end product.

Particularly suitable as marker substances are the rare-earth metals (lanthanides), yttrium, zinc, molybdenum, tungsten, germanium, tin and scandium.

One or more marker substances can be used.

The marker substances can be added at various points of the TiO2 production process. It is preferable that the marker substance be bound so firmly to the TiO2 surface that it is not detached by washing or other subsequent processing steps, and that it be uniformly distributed in the pigment volume.

In one embodiment of the invention, the marker substance(s) is/are added prior to calcining in the framework of the sulphate process.

The method for adding the marker substances preferably fits in with the surface treatment method used in the specific case. Aqueous surface treatment customarily starts with an aqueous titanium dioxide base material suspension, which is first milled in a stirred mill and to which the solution of a corresponding water-soluble salt of the coating substance is then added, e.g. the corresponding sulphate, nitrate, phosphate or chloride, etc. As a result of appropriate pH value control, a hydrated oxide or a phosphate is deposited on the titanium dioxide surface. The pigment is subsequently separated by filtration, freed of water-soluble salts by washing, dried and steam-milled. In this method, the marker substance can be added to the suspension as an appropriate quantity of a water-soluble compound and precipitated at a given pH value. It is likewise possible to add the marker substance during steam milling or during drying. Similarly, one or several different marker substances can be added at different points of the TiO₂ production process. Moreover, the marker substance can also be added as an atomised suspension, or dissolved in an organic solvent, e.g. together with silicone oil treatment in the steam mill.

Surface treatment of titanium dioxide base material in the gas phase is usually performed directly following formation of the TiO₂ base material particles from a gas containing TiCl₄ and oxygen, at temperatures customarily exceeding 800° C. The coating substance is fed into the reactor as a gaseous compound at a point where the TiO₂ formation reaction has almost been completed. It forms a uniform oxide skin on the particle surface. These surface treatment methods are described in, for example, WO 96/36441, EP 0 767 759 B1 and WO 01/81480 A2. The TiO₂ particles are subsequently cooled, separated from the gaseous suspension, additionally subjected to aqueous surface treatment where appropriate, dried and finally steam-milled. For addition of the marker substance during surface treatment in the gas phase, use is preferably made, according to the invention, of a compound of the marker substance that is gaseous at temperatures >800° C., e.g. the corresponding halide. The compound of the marker substance is fed into the reactor together with, or in the direct vicinity of, the other coating substances and is likewise precipitated on the particle surface in the form of an oxide. It is likewise possible to add the marker substance during steam milling or during drying. Similarly, one or several different marker substances can be added at different steps in the process.

The marker substance can also be used as the sole surface treatment component. In this case, the marker substance(s) is/are preferably added during steam milling and/or during drying and/or during calcining in the case of the sulphate process.

The concentration of the marker substance used is preferably minimised, for cost reasons and in order to make detection more difficult for third parties. Detection is dependent on the actual concentration of the substance in the end product and on the detection limit of the detection method to be used. The marker substance is preferably present in a concentration of up to 1% by weight, particularly 0.005 to 0.1% by weight, particularly preferably 0.01 to 0.1% by weight, referred to TiO₂.

In order to be able to achieve reliable, reproducible analysis results, the person skilled in the art will target a minimum concentration corresponding to the detection limit of the substance in question in the sample to be analysed. As the pigment is usually only present in the end product in low concentrations of a few percent, it is in most cases necessary to increase the concentration of the sample of the end product to be analysed in terms of TiO₂ and marker substance. Application

The method according to the invention, for identifying and verifying of products containing titanium dioxide pigment particles, is suitable for use in, for example, the fields of fibres for textiles, papers for documents and currency, in the pharmaceutical sector, and also for plastics, paints and coatings.

In the textile fibres sector, marked TiO2 pigments can, for example, be used to mark special, high-quality fibres, and the products made of them, and protect them against imitation. In addition, manufacturers of high-quality, branded garments can identify fakes by using fibre fabrics delustered with correspondingly marked TiO2 pigments in their products. As a fibre delustering agent, TiO2 is usually used in the range of 0.3 to 1% by weight. If the pigment is coated with roughly 100 ppm to 1,000 ppm marker substance, the latter is contained in the fibre in a concentration of 0.3 ppm to 10 ppm.

High-quality papers contain TiO₂ pigment in the pulp or in the coating, usually in a concentration of up to 10% by weight. Marked TiO₂ pigment can be used in the production of special papers, e.g. for identity cards, certificates or bank notes, on the one hand in order to identify forgeries and, on the other hand, to track the distribution channels of the papers—even of individual batches—for example.

TiO₂ pigment is also used in pharmaceuticals, as a brightener in tablets or for pigmenting the surface coating of medications in the form of coated tablets, usually in quantities of up to roughly 3% by weight, referred to the coating. With the help of the marking according to the invention, fakes can be distinguished from the original pharmaceuticals, and it is furthermore again possible to track the distribution channels, even of individual production batches.

In the plastics sector, the marking of the TiO₂ pigment can help the pigment manufacturer to distinguish between its own pigments and third-party pigments in the plastic end product, and to defend itself against unjustified compensation claims in connection with complaints, e.g. relating to discolouration of PVC window profiles.

In the field of paints and coatings, too, e.g. in vehicle refinish paints or other high-quality coatings, the pigment manufacturer can identify its own and third-party pigments with the help of the pigment marking.

The method according to the invention for marking titanium dioxide pigment particles offers the following advantages:

Use of the marking method presents no problems, since no additional process steps or equipment are necessary during TiO₂ production other than the steps according to the present invention.

Methods for detecting the marker substances of the present invention are established.

The additional costs for TiO₂ production are low.

The present invention also includes a method involving testing a product to determine whether it contains the titanium dioxide pigment particles produced by the method of this invention. In addition, the step of assessing the test results from the test can be used to determine whether the product is the product containing the titanium dioxide pigment particles produced by the method of this invention. Thus, the methods of this invention can be used to verify authentic product and to identify product forgeries (product piracy), to track distribution channels, or to identify the pigment used in order to counter unjustified complaints. One such method for marking titanium dioxide pigment particles and for verifying marked titanium dioxide pigment particles includes the steps of: using a quantity of at least one marker substance during production of the titanium dioxide particles, wherein the marker substance is detectable in a product containing the titanium dioxide particles; recording information about the marker substance; testing a product to determine whether it contains the marker substance; and comparing the results of the test to the recorded information to determine whether the product contains the marker that was used during the production of the titanium dioxide particles.

The invention is explained in detail on the basis of the example below, without this in any way restricting the invention.

### EXAMPLE

A suspension of dry-milled anatase produced by the sulphate process, with a TiO₂ concentration of 500 g/l, is mixed at 60° C. with 0.1% sodium hexametaphosphate as dispersant and set to a pH value of 10 with NaOH. While stirring, 1.0% by weight SiO₂, referred to TiO₂, is added to the suspension in the form of sodium waterglass. HCl is then used to set the pH value to 7. While stirring, 300 ppm La₂O₃, referred to TiO₂, are next added to the suspension in the form of lanthanum sulphate, and the pH value is again set to 7 with NaOH or HCl. Subsequently, 2.0% by weight Al₂O₃, referred to TiO₂—added in the form of sodium aluminate—is precipitated onto the pigment particles at a pH value of 6.5 to 7.5 by means of a fixed-pH method using HCl.

The suspension is set to a pH value of 6.8, freed of water-soluble salts by washing, and dried. Treatment with silicone oil is performed during final steam-milling.

The TiO₂ pigment marked with lanthanum oxide is used for delustering polyester fibres in a concentration of 0.5% by weight TiO₂. A TiO₂ suspension in ethylene glycol is used in this context. The ethylene glycol is reacted with dimethyl terephthalate, the methanol formed is distilled off, and the liquid polyester is withdrawn from the reaction vessel. Filaments with a fibre diameter of a few μm are produced from the delustered polyester strands in further processing steps, and further intermediate steps, such as spinning, weaving, etc., are performed to produce a polyester fabric, e.g. for high-quality textiles, such as net curtains.

To analyse the marker substance in the polyester fibre, 10 g polyester fibre are incinerated at roughly 800° C. The residue is decomposed in a potassium carbonate melt, and the melt dissolved in hydrochloric acid after cooling. The lanthanum is measured by ICP-AES at a wavelength of 408.672 nm, and the concentration determined via a calibration function. The La₂O₃ concentration is 1.5 ppm, referred to the polyester fibre.

1. A method for marking titanium dioxide pigment particles comprising:
using a quantity of at least one marker substance during production of the titanium dioxide particles, wherein the marker substance is detectable in a product containing the titanium dioxide particles.

2. The method according to claim 1, further comprising:
adding said at least one marker substance so that the titanium dioxide pigment particle displays a quantity of up to 1% by weight of said marker substance.

3. The method according to claim 2, wherein the step of adding said at least one marker substance produces a titanium dioxide pigment particle having from about 0.005% to about 0.1% by weight of said marker substance.

4. The method according to claim 1 wherein
said at least one marker substance is selected from the group comprising: the lanthanides; yttrium; zinc; molybdenum; tungsten; germanium; tin; and scandium.

5. The method according to claim 4 wherein said at least one marker substance is selected from the group comprising: zinc; cerium; yttrium; and molybdenum.

6. The method according to claim 1, further comprising: adding said at least one marker substance in the TiO₂ production process during calcining.

7. The method according to claim 1, further comprising: adding said at least one marker substance in the TiO₂ production process during aqueous or gas-phase surface treatment.

8. The method according to claim 1, further comprising: adding said at least one marker substance in the TiO₂ production process during drying.

9. The method according to claim 1, further comprising: adding said at least one marker substance in the TiO₂ production process during steam-milling.

10. The method according to claim 1, further comprising: adding said at least one marker substance in the TiO₂ production process after steam-milling.

11. The Titanium dioxide pigment particles produced by the method according to claim 1.

12. Using the Titanium dioxide pigment particles of claim 11 in paper.

13. Using the Titanium dioxide pigment particles of claim 11 in textiles fibres.

14. Using the Titanium dioxide pigment particles in claim 11 in plastics.

15. Using the Titanium dioxide pigment particles of claim 11 in coatings.

16. Using the Titanium dioxide pigment particles of claim 11 in pharmaceuticals.

17. A method for marking a product containing titanium dioxide pigment particles comprising: using the titanium dioxide pigment particles produced by the method of claim 1.

18. Product comprising the titanium dioxide pigment particles produced by the method of claim 1.

19. A method using the titanium dioxide pigment particles produced by the method of claim 1 comprising:
testing a product to determine whether it contains the titanium dioxide pigment particles produced by the method of claim 1.

20. The method of claim 19 further comprising:
assessing the test results from said test to determine whether said product is the product containing the titanium dioxide pigment particles produced by the method of claim 1.

21. A method for marking titanium dioxide pigment particles and verifying titanium dioxide pigment particles comprising:
using a quantity of at least one marker substance during production of the titanium dioxide particles, wherein the marker substance is detectable in a product containing the titanium dioxide particles;
recording information about the marker substance;
testing a product to determine whether it contains the marker substance; and comparing the results of the test to the recorded information to determine whether the product contains the marker that was used during the production of the titanium dioxide particles.
